(19) United States
(12) Patent Application Publication
(10) Pub. No.: US 2025/0312026 A1
(43) Pub. Date: Oct. 9, 2025

(54) ANCHORING DEVICE WITH FINGER SECURING PROTRUSIONS AND METHOD FOR INSERTION THEREOF

(71) Applicant: FEMSelect Ltd., Modi'in (IL)

(72) Inventors: Boaz Harari, Ganei Tikvah (IL); Vincent Robert Lucente, Macungie, PA (US)

(73) Assignee: FEMSelect Ltd., Modi'in (IL)

(21) Appl. No.: 18/629,318

(22) Filed: Apr. 8, 2024

(51) Int. Cl. A61B 17/04; A61B 17/00; A61B 17/42; A61B 42/20; A61B 90/00

(52) U.S. Cl. CPC A61B 17/0401; A61B 17/00234; A61B 17/42; A61B 42/20; A61B 90/08; A61B 2017/00265; A61B 2017/00367; A61B 2017/0046; A61B 2017/00743; A61B 2017/0409

(57) ABSTRACT

An anchoring device, including a finger-mountable work channel defining assembly defining two side walls joined at an end wall, the finger-mountable work channel defining assembly defining a finger mounting axis, the finger-mountable work channel defining assembly defining a work channel axis along one of the side walls, the finger-mountable work channel defining assembly defining an aperture lying generally in an aperture plane angled with respect to the finger mounting axis and with respect to the work channel axis, the finger-mountable work channel defining assembly defining at least one protrusion adjacent to the aperture, an anchor insertion assembly arranged for axial displacement along the work channel axis with respect to the aperture so as to intersect the aperture plane and an anchor assembly including at least an anchoring portion and a flexible elongate portion attached thereto, the anchoring portion having a pre-anchoring operative orientation and an anchoring operative orientation.

### REFERENCE TO RELATED APPLICATIONS

Reference is hereby made to the Applicant's following patents and patent applications, which are related to the subject matter of the present application, the disclosures of which are hereby incorporated by reference:
U.S. Pat. No. 9,517,058 entitled SYSTEM AND METHOD FOR PELVIC FLOOR REPAIR;
U.S. Patent Publication No. 2015/0320442 A1 entitled SYSTEM AND METHOD FOR PELVIC FLOOR PROCEDURES;
U.S. Patent Publication No. 2017/0196671 A1 entitled TISSUE REPAIR DEVICE AND METHOD; and
U.S. Patent Publication No. 2021/0128136 A1 entitled ANCHORING DEVICE AND METHOD FOR ACCURATE POSITIONING AND INSERTION OF AN ANCHOR ASSEMBLY.

### FIELD OF INVENTION

The present invention relates to anchoring devices generally.

### BACKGROUND OF THE INVENTION

Various types of anchoring devices are known for various applications.

### SUMMARY OF THE INVENTION

The present invention seeks to provide improved apparatus for the accurate insertion of an anchoring assembly into an anchoring substrate.

There is thus provided in accordance with a preferred embodiment of the present invention an anchoring device, including a finger-mountable work channel defining assembly defining two side walls joined at an end wall, the finger-mountable work channel defining assembly defining a finger mounting axis, the finger-mountable work channel defining assembly defining a work channel axis along one of the side walls, the finger-mountable work channel defining assembly defining an aperture lying generally in an aperture plane angled with respect to the finger mounting axis and with respect to the work channel axis, the finger-mountable work channel defining assembly defining at least one protrusion adjacent to the aperture, an anchor insertion assembly arranged for axial displacement along the work channel axis with respect to the aperture so as to intersect the aperture plane and an anchor assembly including at least an anchoring portion and a flexible elongate portion attached thereto, the anchoring portion having a pre-anchoring operative orientation and an anchoring operative orientation.

Preferably, the at least one protrusion is a tooth having a generally tapered shape.

Preferably, the at least one protrusion extends in an axial direction with respect to the aperture.

Preferably, the anchor insertion assembly is arranged for limited axial displacement along the work channel axis with respect to the aperture so as to intersect the aperture plane.

Preferably, the anchoring portion of the anchoring device is displaceable along the work channel axis across the aperture plane by the anchor insertion assembly while in the pre-anchoring operative orientation.

Preferably, the finger mounting axis lies in a finger mounting axis plane, the work channel axis lies in a work channel axis plane, which is parallel to the finger mounting axis plane and the aperture plane is orthogonal to the mutually parallel finger mounting axis plane and to the work channel axis plane. Alternatively, the work channel axis is angled with respect to the finger mounting axis.

Preferably, the finger-mountable work channel defining assembly defines a finger anchoring location engagement portion lying generally in an orientation angled with respect to the work channel axis.

The finger-mountable work channel defining assembly may be configured to be either right- or left-hand specific.

Preferably, the finger-mountable work channel defining assembly includes a finger mount element, an elongate work channel defining element and an insertion and stop defining element, which is fixedly mounted onto the elongate work channel defining element.

Preferably, the finger-mountable work channel defining assembly is formed as a single piece of a partially flexible plastic material and is configured to enable an underside of an operator's finger to engage an anchoring site.

Preferably, the work channel defining assembly defines forward and rearward throughgoing fixed mounting channels for fixedly mounting the elongate work channel defining element.

Preferably, the insertion and stop defining element is a hollow element, which includes a generally cylindrical hollow portion which communicates with a hollow funnel-shaped portion defining an edge, which serves as a precise stop limiting the axial element of displacement of the anchor insertion assembly therethrough.

Preferably, the anchor insertion assembly includes first and second housing portions onto which are mounted an inner tube and button assembly including a button and an elongate tube fixedly mounted thereto at an end of the elongate tube.

In accordance with a preferred embodiment of the present invention, the anchoring device further includes a button guard element pivotably mounted onto the first and second housing portions for selectable protective engagement with the button.

There is also provided in accordance with a preferred embodiment of the present invention a method for utilizing an anchoring device for positioning and inserting an anchoring assembly into an anchoring substrate, the anchoring device including a finger-mountable work channel defining assembly defining two side walls joined at an end wall, the finger-mountable work channel defining assembly defining a finger mounting axis, the finger-mountable work channel defining assembly defining a work channel axis along one of the side walls, the finger-mountable work channel defining assembly defining an aperture lying generally in an aperture plane angled with respect to the finger mounting axis and with respect to the work channel axis, the finger-mountable work channel defining assembly defining at least one protrusion adjacent to the aperture, an anchor insertion assembly arranged for axial displacement along the work channel axis with respect to the aperture so as to intersect the aperture plane, and an anchor assembly including at least an anchoring portion and a flexible elongate portion attached thereto, the anchoring portion having a pre-anchoring operative orientation and an anchoring operative orientation, the method including inserting a finger of an operator into the finger-mountable work channel defining assembly, whereby a distal phalanx of the finger is arranged so as to engage the anchoring substrate, engaging the at least one protrusion with the anchoring substrate to prevent slip of the finger, and inserting the anchor insertion assembly into the finger-mountable work assembly, with the anchor assembly preloaded therein, along the work channel axis, such that the orientation of the finger of the an operator precisely defines the location of anchoring engagement of the anchor assembly with the anchoring substrate.

Preferably, an extent of insertion of the anchor assembly in the anchoring substrate is precisely limited by engagement between the anchor insertion assembly and the finger-mountable work channel defining assembly.

### BRIEF DESCRIPTION OF THE DRAWINGS

FIGS. 1A and 1B are simplified schematic exploded-view illustrations of an anchoring device including a finger-mountable work channel defining assembly defining a finger aperture and protrusions adjacent to the finger aperture, an anchor insertion assembly and an anchor assembly, in a first and second operative orientation, respectively;

FIGS. 2A and 2B are simplified schematic respective bottom-view isometric and planar illustrations of the finger-mountable work channel defining assembly of FIGS. 1A & 1B, in the operative orientation of FIG. 1A;

FIGS. 2C and 2D are simplified schematic respective bottom-view isometric and planar illustrations of the finger-mountable work channel defining assembly of FIGS. 1A & 1B, in the operative orientation of FIG. 1B;

FIG. 3A is a simplified schematic isometric illustration of the anchoring device of FIGS. 1A-2D, in a first pre-anchoring operative orientation;

FIG. 3B is a simplified schematic isometric illustration of the anchoring device of FIGS. 1A-3A, in a second pre-anchoring operative orientation;

FIGS. 3C and 3D are simplified schematic respective isometric and side-planar illustrations of the anchoring device of FIGS. 1A-3B, in an anchoring operative orientation;

FIGS. 4A and 4B are simplified schematic respective bottom-view isometric and planar illustrations of a portion of the finger-mountable work channel defining assembly of FIGS. 1A-3D, showing a first arrangement of protrusions; and

FIGS. 5A and 5B are simplified schematic respective bottom-view isometric and planar illustrations of an additional embodiment of a portion of a finger-mountable work channel defining assembly similar to that of FIGS. 1A-4B, showing an alternative arrangement of protrusions.

### DETAILED DESCRIPTION OF PREFERRED EMBODIMENTS

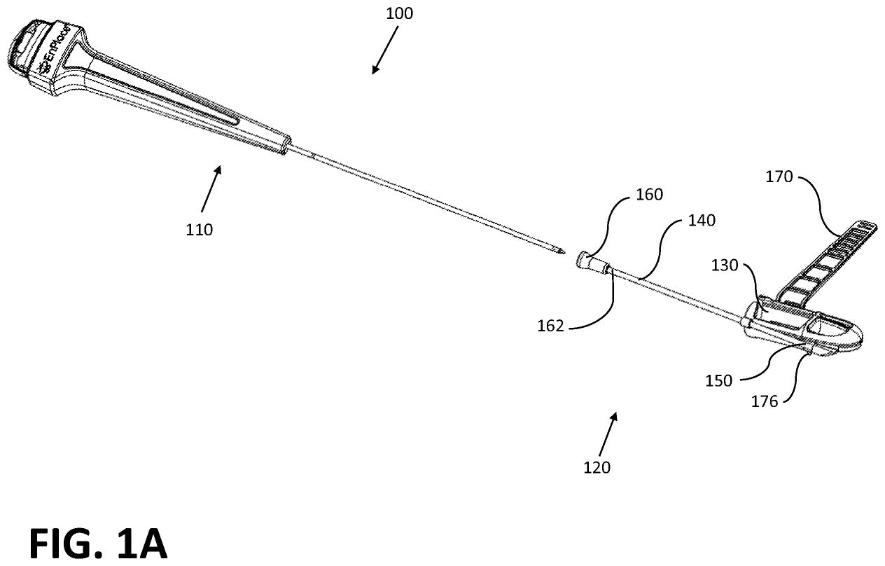

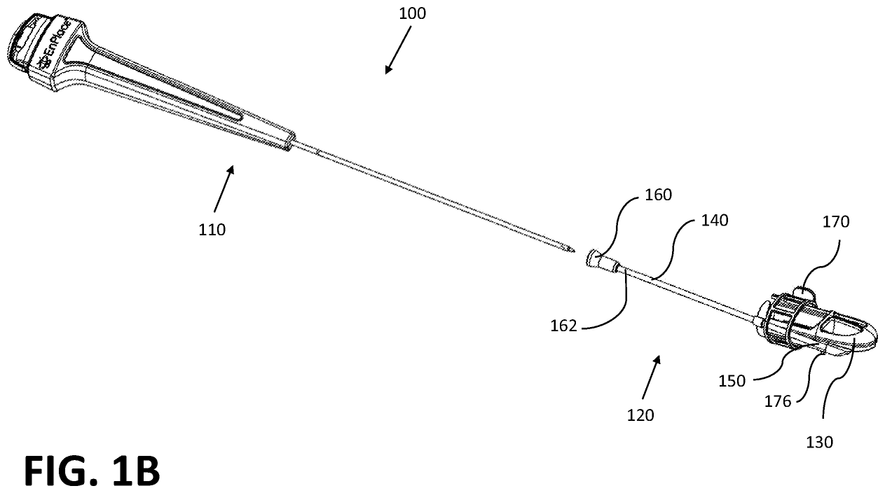

Reference is now made to FIGS. 1A and 1B, which are simplified schematic exploded-view illustrations of an anchoring device 100, in a first and second operative orientation, respectively. Anchoring device 100 preferably includes an anchor insertion assembly 110 and a finger-mountable work channel defining assembly 120. Finger-mountable work channel defining assembly 120 preferably includes a finger mount element 130 and an elongate work channel defining element 140. Elongate work channel defining element 140 is preferably fixedly mounted, at a first end 150 thereof, onto finger mount element 130.

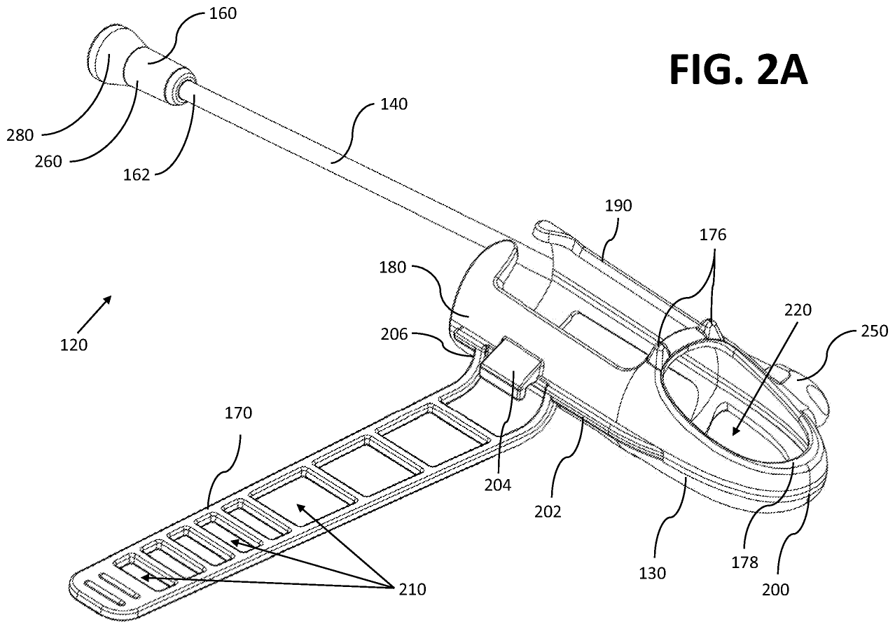

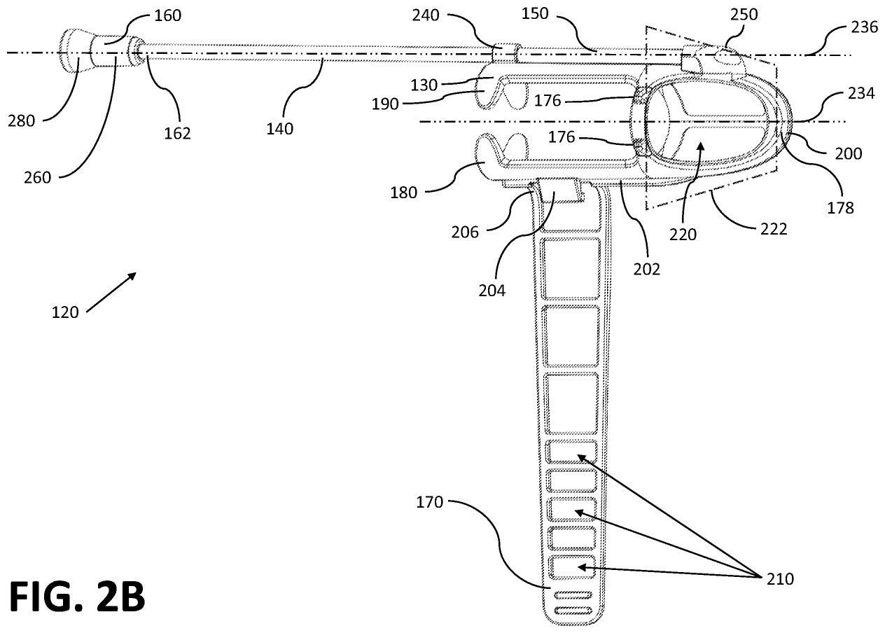

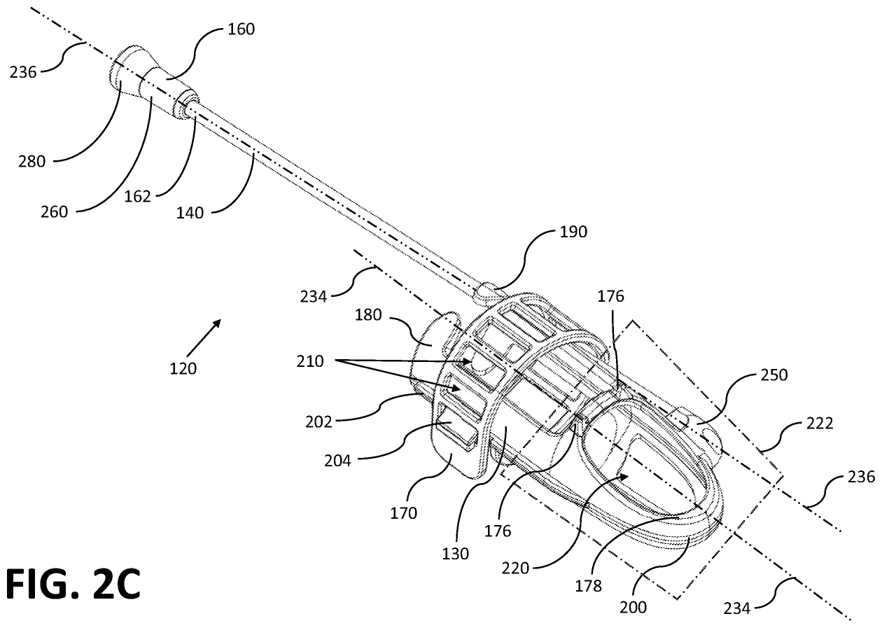

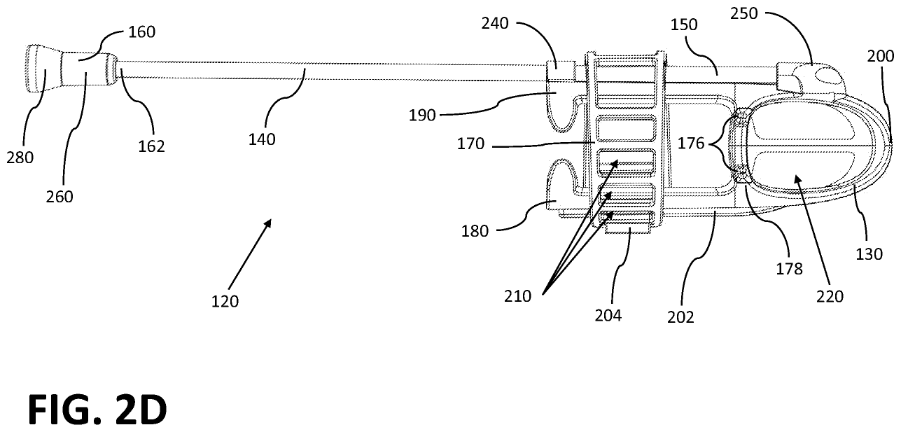

Finger-mountable work channel defining assembly 120 preferably additionally includes an insertion and stop defining element (ISDE) 160, which is preferably fixedly mounted onto elongate work channel defining element 140 at a second end 162 of work channel defining element 140. Finger-mountable work channel defining assembly 120 preferably further includes an adjustable strap 170, described hereinbelow with particular reference to FIGS. 2A-2D.

Finger-mountable work channel defining assembly 120, and preferably finger mount element 130 thereof, defines at least one protrusion 176, and preferably a multiplicity of protrusions 176, which extend radially outwardly from a lower wall 178 of finger mount element 130. The function of protrusions 176 is to engage with an anchoring substrate and hold an operator's finger in place and prevent slip, particularly when an operator's finger is searching for and engaging a potential anchoring site. It is appreciated that protrusions 176 assist in preventing slip particularly of an operator's finger that is inserted into finger-mountable work channel defining assembly 120.

Reference is now made to FIGS. 2A-2D. FIGS. 2A and 2B are simplified schematic respective bottom-view isometric and planar illustrations of finger-mountable work channel defining assembly 120, in the operative orientation of FIG. 1A. FIGS. 2C and 2D are simplified schematic respective bottom-view isometric and planar illustrations of finger-mountable work channel defining assembly 120, in the operative orientation of FIG. 1B.

Finger mount element 130 is preferably configured to removably accommodate an index finger of an operator, and is preferably formed of a single piece of partially flexible material, such as, inter alia, polycarbonate. In addition to lower wall 178, finger mount element 130 preferably defines a first side wall 180 and a second side wall 190, which are preferably joined at an end wall 200. First side wall 180 preferably includes a strap-mounting feature 202 and a strap-closure protrusion 204.

Adjustable strap 170 includes a mounting end 206, which is preferably mounted to strap-mounting feature 202 on first side wall 180 of finger mount element 130. Adjustable strap 170 preferably further includes a multiplicity of fastening apertures 210. To close adjustable strap 170, thereby securing a finger of an operator within finger mount element 130, adjustable strap 170 is looped around an outer surface of finger mount element 130, and a suitable one of fastening apertures 210 is preferably secured to strap-closure protrusion 204, as seen particularly in FIGS. 2C & 2D.

Preferably, when strap 170 is in a closed operative orientation, as seen particularly in FIGS. 2C & 2D, strap 170 exerts a radially inwardly-directed force on both first and second side walls 180 and 190 of finger mount element 130, thereby securing a finger of an operator therewithin. In a preferred embodiment of the present invention, when strap 170 is in a closed operative orientation, strap 170 exerts a radially inwardly-directed force on substantially an entire circumference of a portion of an operator's finger, thereby securing the operator's finger within finger mount element 130.

Finger mount element 130, particularly lower wall 178 thereof, preferably further includes a finger aperture 220, which preferably lies generally in an aperture plane 222. Finger aperture 220 is configured to enable an underside of an operator's finger, when properly inserted into finger mount element 130 of finger-mountable work channel defining assembly 120, to be exposed and to engage a potential anchoring site. A preferable underside of an operator's finger is a pad of an operator's finger, more preferably a pad of an operator's index finger. Thus, in a preferred method of use of anchoring device 100, an operator inserts his or her finger into finger-mountable work channel defining assembly 120, such that a distal phalanx of the operator's finger is arranged so as to engage an anchoring substrate.

In the embodiment of the present invention shown in FIGS. 1A-4B, finger-mountable work channel defining assembly 120 is configured to accommodate a left index finger of an operator, and thus is left hand specific. In an alternative embodiment of the present invention, finger-mountable work channel defining assembly 120 is configured to accommodate a right index finger of an operator, and thus is right hand specific.

As seen particularly in FIGS. 2B and 2C, finger mount element 130 preferably defines and is generally coaxial with a finger mounting axis 234, and elongate work channel defining element 140 preferably defines and is generally coaxial with a work channel axis 236. Aperture plane 222 is preferably angled with respect to finger mounting axis 234 and with respect to work channel axis 236. As seen particularly in FIG. 2C, work channel axis 236 is preferably angled with respect to finger mounting axis 234. Thus, finger-mountable work channel defining assembly 120 defines a finger anchoring location engagement portion, such as finger aperture 220 and protrusions 176, lying generally in an orientation, such as that of aperture plane 222, which is angled with respect to work channel axis 236.

As described hereinabove with reference to FIGS. 1A & 1B, finger mount element 130 is formed with at least one protrusion 176, and more preferably with two or more protrusions 176, which extend radially outwardly from lower wall 178 of finger mount element 130. Protrusions 176 are preferably adjacent to finger aperture 220. In the embodiment of the present invention shown in FIGS. 1A-4B, finger mount element 130 includes two protrusions 176. In an alternative embodiment of the present invention, finger mount element 130 defines any suitable number of protrusions 176, such as one protrusion 176, two protrusions 176, three protrusions 176, four protrusions 176, five protrusions 176, six protrusions 176, or more than six protrusions 176.

In the embodiment of the present invention shown in FIGS. 1A-4B, protrusions 176 are teeth with generally tapered shapes. In an alternative embodiment of the present invention, protrusions 176 are of any suitable shape, such as, inter alia, generally spherical beads. In the embodiment of the present invention shown in FIGS. 1A-4B, protrusions 176 extend radially outwardly from lower wall 178, and are generally perpendicular to finger aperture 220. In an alternative embodiment of the present invention, protrusions 176 extend in an axial direction or tangential direction with respect to finger aperture 220. Protrusions 176 may have any suitable shape and size, and are not limited by the relative lengths and widths with respect to finger mount element 130, shown in FIGS. 1A-4B.

As seen particularly in FIGS. 2B and 2D, finger mount element 130, preferably second side wall 190 thereof, preferably includes respective forward and rearward throughgoing fixed mounting channels 240 and 250 for fixedly mounting elongate work channel defining element 140 onto finger mount element 130. Elongate work channel defining element 140 is preferably fixed to finger mount element 130 within mounting channels 240 and 250 using a suitable adhesive.

Elongate work channel defining element 140 is preferably an elongate hollow tube operative to receive a portion of anchor insertion assembly 110. Elongate work channel defining element 140 is preferably formed of stainless steel and preferably has an inner diameter of 2.6 mm. ISDE 160 is preferably a hollow element including a generally cylindrical hollow portion 260, which accommodates second end 162 of elongate work channel defining element 140, which is preferably fixed thereto using a suitable adhesive. Preferably, generally cylindrical hollow portion 260 communicates with a hollow frustrum, such as a funnel-shaped portion 280, defining an edge, which serves as a precise stop, limiting an axial extent of displacement of anchor insertion assembly 110 through ISDE 160 along work channel axis 236.

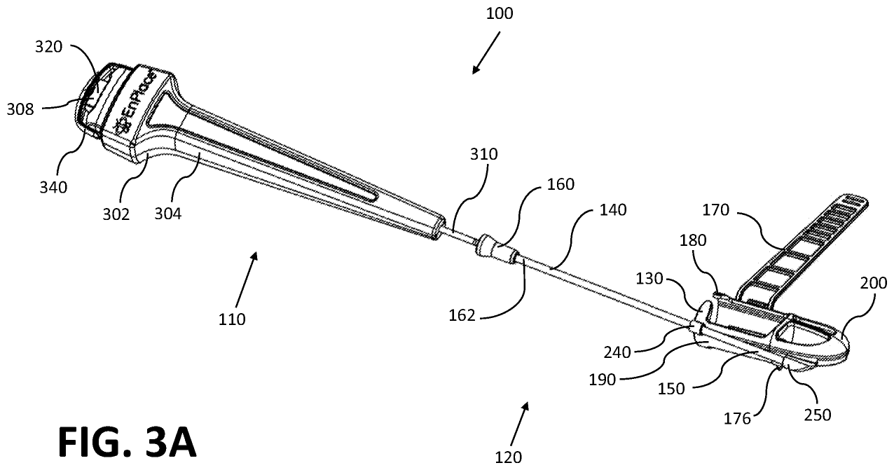

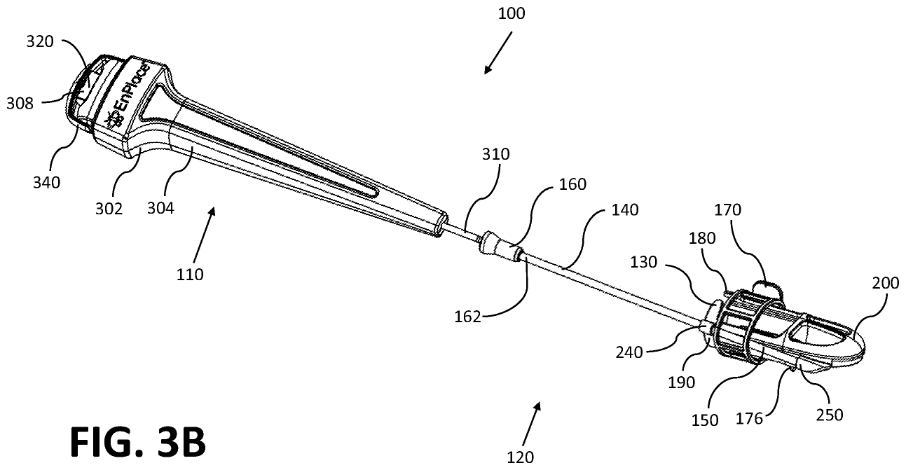

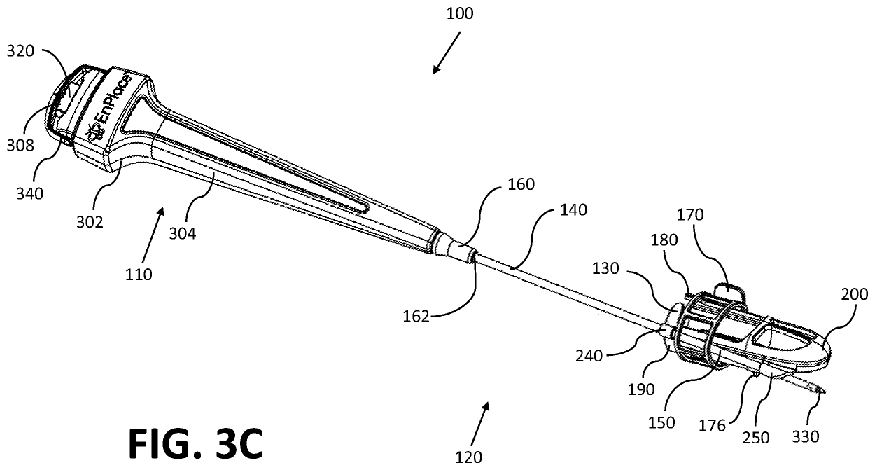

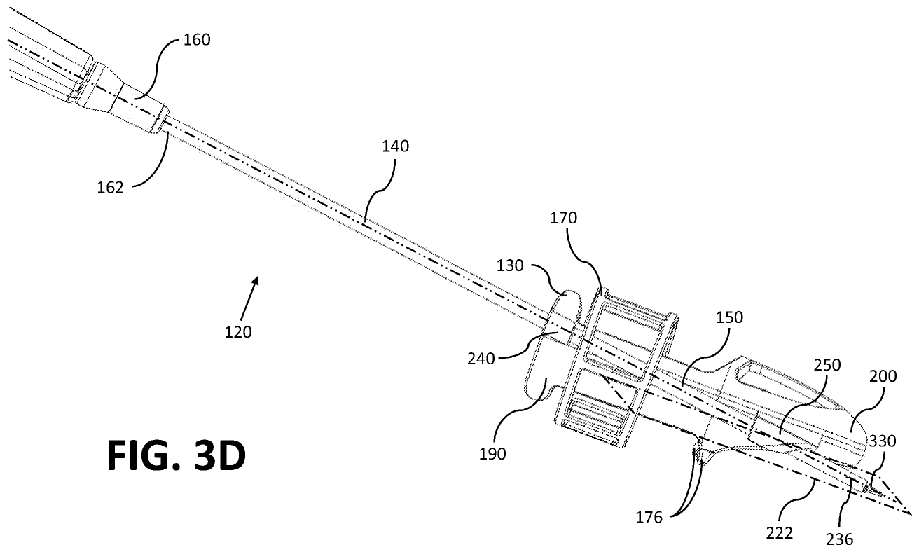

Reference is now made to FIGS. 3A-3D. FIG. 3A is a simplified schematic isometric illustration of anchoring device 100 in a first pre-anchoring operative orientation, wherein strap 170 is in an opened operative orientation. FIG. 3B is a simplified schematic isometric illustration of anchoring device 100 in a second pre-anchoring operative orientation, wherein strap 170 is in a closed operative orientation. FIGS. 3C and 3D are simplified schematic respective isometric and side-view illustrations of anchoring device 100 in an anchoring operative orientation.

Anchor insertion assembly 110 preferably includes a first housing portion 302 and a second housing portion 304, onto which are mounted an inner tube (not shown) and a button assembly 308. Button assembly 308 preferably includes an elongate tube 310, which is preferably threaded through work channel defining element 140 via ISDE 160, and a button 320. An end of elongate tube 310 is preferably fixedly mounted to button 320. When button 320 is depressed, button 320 preferably deploys an anchor assembly 330 at an end of elongate tube 310, as seen particularly in FIGS. 3C and 3D.

In a preferred embodiment of the present invention, anchor assembly 330 includes at least an anchoring portion and a flexible elongate portion attached thereto. As seen particularly in FIG. 3D, anchor insertion assembly 110 is preferably arranged for axial displacement along work channel axis 236 with respect to finger aperture 220 so as to intersect aperture plane 222 when anchoring device 100 is in an anchoring operative orientation. More preferably, anchor insertion assembly 110 is arranged for limited axial displacement along work channel axis 236 with respect to finger aperture 220 so as to intersect aperture plane 222. Axial displacement of anchor insertion assembly 110 along work channel axis 236 is preferably limited by ISDE 160. As described hereinabove, the edge of funnel-shaped portion 280 serves as a precise stop, limiting an axial extent of displacement of anchor insertion assembly 110 through ISDE 160 along work channel axis 236.

It is appreciated that in the pre-anchoring operative orientations shown in FIGS. 3A and 3B, anchoring portion 330 is in a pre-anchoring operative orientation, and in the anchoring operative orientation shown in FIGS. 3C and 3D, anchoring portion 330 is in an anchoring operative orientation.

In the embodiment of the present invention seen particularly in FIGS. 3A and 3B, button 320 is selectively protected with a button guard element 340. In an alternative embodiment of the present invention, button 320 is not selectively protected with a button guard element 340. Preferably, button guard element 340 is pivotably mounted onto first and second housing portions 302 and 304 for selectable protective engagement with button 320. In other words, by pivoting button guard element 340, a user can selectably either place button guard element 340 in protective engagement with button 320 or disengage button guard element 340 from button 320, thereby allowing button 320 to be depressed with relative case.

Preferably, an underside of an operator's finger, such as a pad of a distal phalanx, engages with potential anchoring sites through finger aperture 220, allowing the operator to locate a suitable anchoring site by tactile feedback. When the operator ascertains, preferably at least partially through tactile feedback, that anchoring device 100 is suitably positioned with respect to a suitable anchoring site, the operator depresses button 320, thereby deploying anchor assembly 330. Preferably, protrusions 176 assist in preventing undesired motion of work channel defining assembly 120, and the operator's finger located therein, throughout the deployment of anchor assembly 330. More specifically, protrusion or protrusions 176 preferably mechanically engage with an anchoring substrate, thereby preventing slip of an operator's finger, particularly during a deployment of anchor assembly 330.

In a preferred method of use of anchoring device 100, anchor assembly 330 is preloaded within anchor insertion assembly 110, and anchor insertion assembly 110 is inserted into finger-mountable work channel defining assembly 120 along work channel axis 236. Furthermore, an orientation of an operator's finger that has been inserted into finger-mountable work channel defining assembly 120 preferably precisely defines a location of anchoring engagement of anchor assembly 330, upon deployment thereof, with the anchoring substrate.

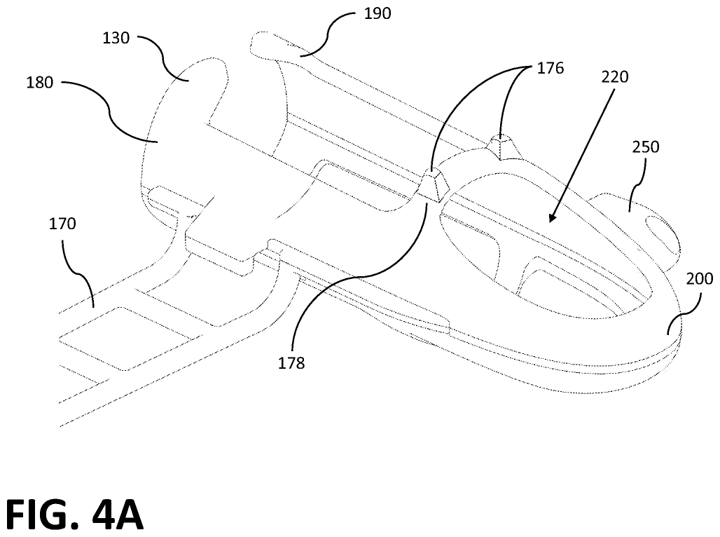

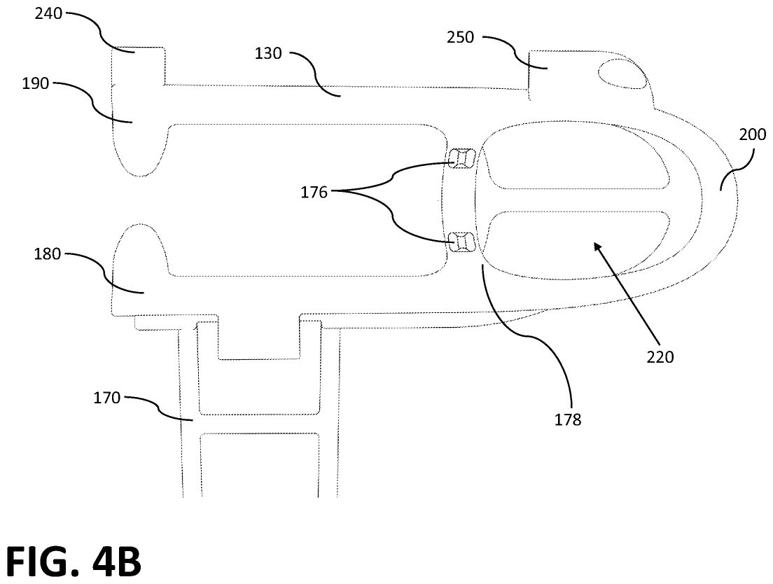

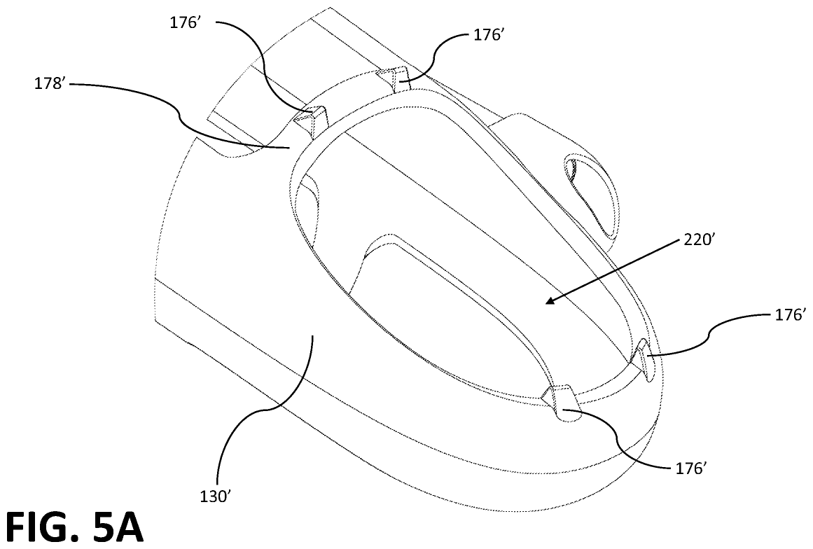

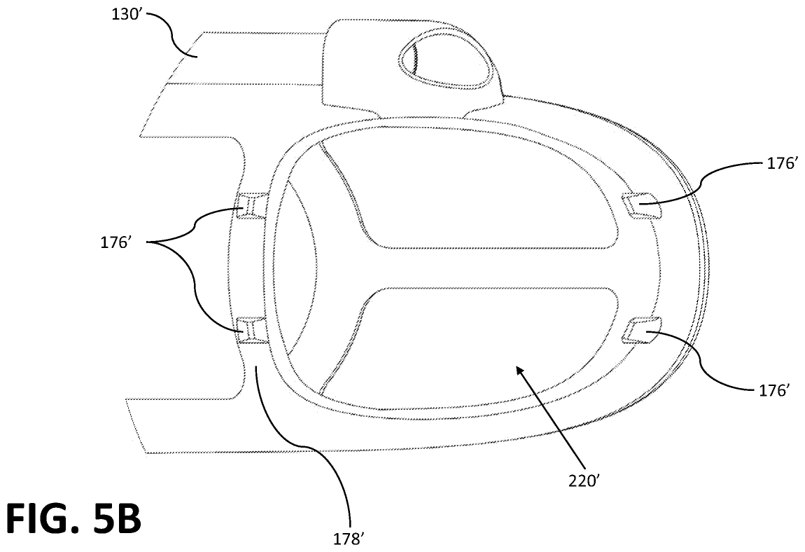

Reference is now made to FIGS. 4A and 4B, which are simplified schematic respective bottom-view isometric and planar illustrations of a portion of finger-mountable work channel defining assembly 120, showing a first arrangement of protrusions 176. In the embodiment of the present invention shown in FIGS. 1A-4B, protrusions 176 are adjacent to finger aperture 220. In the embodiment of the present invention shown in FIGS. 1A-4B, protrusions 176 are on a proximal portion of lower wall 178 of finger mount element 130, between ISDE 160 and finger aperture 220. However, it is appreciated that 100 may include any suitable number or arrangement of protrusions 176, such as, inter alia, those of an alternative embodiment of the present invention shown in FIGS. 5A and 5B.

Reference is now made to FIGS. 5A and 5B, which are simplified schematic respective bottom-view isometric and planar illustrations of an additional embodiment of a portion of a finger-mountable work channel defining assembly similar to finger-mountable work channel defining assembly 120, shown in FIGS. 1A-4B. It is appreciated that corresponding features of the embodiments of FIGS. 1A-4B and of FIGS. 5A & 5B are labelled with corresponding reference numbers. For example, finger mount element 130 of FIGS. 1A-4B corresponds to a finger mount element 130′ of FIGS. 5A & 5B.

Finger mount element 130′ includes, inter alia, at least one protrusion 176′, more preferably four protrusions 176′, extending from a lower wall 178′ of finger mount element 130′. Finger mount element 130′ additionally includes a finger aperture 220′. Finger mount element 130′ has an alternative arrangement of protrusions 176′ with respect to that of protrusions 176 of FIGS. 1A-4B.

As seen particularly in FIGS. 5A and 5B, protrusions 176′ are adjacent to finger aperture 220′. In the embodiment of the present invention shown in FIGS. 5A and 5B, each of protrusions 176′ is positioned at a location circumferential to finger aperture 220′ such that two of protrusions 176′ are diametrically opposed to two additional ones of protrusions 176′. In an alternative embodiment of the present invention, each of protrusions 176′ is placed at a different location on the circumference of finger aperture 220′, preferably adjacent to finger aperture 220′.

It will be appreciated by persons skilled in the art that the present invention is not limited to what has been particularly shown and described hereinabove. The scope of the present invention includes both combinations and sub-combinations of various features described hereinabove as well as modifications thereof, all of which are not in the prior art.

1. An anchoring device comprising:
a finger-mountable work channel defining assembly defining two side walls joined at an end wall, said finger-mountable work channel defining assembly defining a finger mounting axis, said finger-mountable work channel defining assembly defining a work channel axis along one of said side walls, said finger-mountable work channel defining assembly defining an aperture lying generally in an aperture plane angled with respect to said finger mounting axis and with respect to said work channel axis,
said finger-mountable work channel defining assembly defining at least one protrusion adjacent to said aperture;
an anchor insertion assembly arranged for axial displacement along said work channel axis with respect to said aperture so as to intersect said aperture plane; and
an anchor assembly including at least an anchoring portion and a flexible elongate portion attached thereto, said anchoring portion having a pre-anchoring operative orientation and an anchoring operative orientation.

2. An anchoring device according to claim 1, wherein said at least one protrusion is a tooth having a generally tapered shape.

3. An anchoring device according to claim 1, wherein said at least one protrusion extends in an axial direction with respect to said aperture.

4. An anchoring device according to claim 1, wherein said anchor insertion assembly is arranged for limited axial displacement along said work channel axis with respect to said aperture so as to intersect said aperture plane.

5. An anchoring device according to claim 1, wherein said anchoring portion is displaceable along said work channel axis across said aperture plane by said anchor insertion assembly while in said pre-anchoring operative orientation.

6. An anchoring device according to claim 1, wherein said finger mounting axis lies in a finger mounting axis plane, said work channel axis lies in a work channel axis plane, which is parallel to said finger mounting axis plane and said aperture plane is orthogonal to said mutually parallel finger mounting axis plane and to said work channel axis plane.

7. An anchoring device according to claim 1, wherein said work channel axis is angled with respect to said finger mounting axis.

8. An anchoring device according to claim 1, wherein said finger-mountable work channel defining assembly defines a finger anchoring location engagement portion lying generally in an orientation angled with respect to said work channel axis.

9. An anchoring device according to claim 1, wherein said finger-mountable work channel defining assembly is configured to be either right or left hand specific.

10. An anchoring device according to claim 1, wherein said finger-mountable work channel defining assembly comprises a finger mount element, an elongate work channel defining element and an insertion and stop defining element, which is fixedly mounted onto said elongate work channel defining element.

11. An anchoring device according to claim 10, wherein said finger-mountable work channel defining assembly is formed as a single piece of a partially flexible plastic material and is configured to enable an underside of an operator's finger to engage an anchoring site.

12. An anchoring device according to claim 10, wherein said finger-mountable work channel defining assembly defines forward and rearward throughgoing fixed mounting channels for fixedly mounting said elongate work channel defining element.

13. An anchoring device according to claim 10, wherein said insertion and stop defining element is a hollow element, which includes a generally cylindrical hollow portion which communicates with a hollow funnel-shaped portion defining an edge, which serves as a precise stop limiting the axial element of displacement of said anchor insertion assembly therethrough.

14. An anchoring device according to claim 1, wherein said anchor insertion assembly comprises first and second housing portions onto which are mounted an inner tube and button assembly including a button and an elongate tube fixedly mounted thereto at an end of said elongate tube.

15. An anchoring device according to claim 14, further comprising a button guard element pivotably mounted onto said first and second housing portions for selectable protective engagement with said button.

16. A method for utilizing an anchoring device for positioning and inserting an anchoring assembly into an anchoring substrate, said anchoring device comprising:
a finger-mountable work channel defining assembly defining two side walls joined at an end wall, said finger-mountable work channel defining assembly defining a finger mounting axis, said finger-mountable work channel defining assembly defining a work channel axis along one of said side walls, said finger-mountable work channel defining assembly defining an aperture lying generally in an aperture plane angled with respect to said finger mounting axis and with respect to said work channel axis,
said finger-mountable work channel defining assembly defining at least one protrusion adjacent to said aperture;
an anchor insertion assembly arranged for axial displacement along said work channel axis with respect to said aperture so as to intersect said aperture plane; and
an anchor assembly including at least an anchoring portion and a flexible elongate portion attached thereto, said anchoring portion having a pre-anchoring operative orientation and an anchoring operative orientation,
said method comprising:
inserting a finger of an operator into said finger-mountable work channel defining assembly, whereby a distal phalanx of said finger is arranged so as to engage said anchoring substrate;
engaging said at least one protrusion with said anchoring substrate to prevent slip of said finger; and
inserting said anchor insertion assembly into said finger-mountable work channel defining assembly, with said anchor assembly preloaded therein, along said work channel axis, such that the orientation of said finger of said operator precisely defines the location of anchoring engagement of said anchor assembly with said anchoring substrate.

17. A method according to claim 16, wherein an extent of insertion of said anchor assembly in said anchoring substrate is precisely limited by engagement between said anchor insertion assembly and said finger-mountable work channel defining assembly.
